(19) United States
(12) Patent Application Publication
(10) Pub. No.: US 2007/0281361 A1
(43) Pub. Date: Dec. 6, 2007

(54) Human plasma free amino acids profile using pre-column derivatizing reagent- 1-naphthylisocyanate and high performance liquid chromatographic method

(76) Inventor: Meenakshisundaram Hariharan, Tarzana, CA (US)

Correspondence Address: MEENAKSHISUNDARAM HARIHARAN, 18806 HATTERAS ST # 106, TARZANA, CA, 91356, US

(21) Appl. No.: 11/803,027

(22) Filed: May 11, 2007

Related U.S. Application Data
(60) Provisional application No. 60/799,401, filed on May 11, 2006.

(51) Int. Cl. G01N 30/02; G01N 30/06; G01N 30/28; G01N 30/50

(52) U.S. Cl. 436/89

(57) ABSTRACT

After many decades, 1-Naphthylisocyanate (NIC) has been identified as the most ideal pre column derivatization fluorescent tag for reversed phase Liquid Chromatographic (HPLC) analysis of all free amino acids (AA) in biological samples. NIC forms very stable derivatives with all AAs in one minute. Using NIC, the first, most simple, robust, sensitive (femto mole), and economical high pressure binary gradient, HPLC method, has been developed. It estimates 35 (and 2 internal standards) AAs in human plasma in record shortest time of 20 minutes and has been validated for precision (n=16, <6%), accuracy 95 %, linearity (0 to 1200 μM/L), and analyzing normal and abnormal patients. It can provide with in 20 minutes the first and best plasma free AAs profile that includes Homocysteine, Cysteine, Alloisoleucine, and Cystathionine and a 27 AAs profile using a blood spot (3 μl plasma). A sample can be analyzed with in one hour of its arrival in the laboratory.

### BACKGROUND

The determination of the concentrations free amino acids in biological samples is a very important clinical diagnostic test. Most of the clinical laboratories perform the assay using the High Performance Liquid Chromatographic (HPLC) technique. There are only two commercial HPLC methods that are widely in use for the past three decades. They are the ion exchange method (about 6 vendors are there around the world) and the “PICO Tag™” method (1) of “Waters” chromatographic company USA. Both methods are Ultraviolet detection methods. The two methods represent two different types of HPLC methods. The ion-exchange method is a post column derivatization method and the PICO tag method is a pre column derivatization method which uses Phenylisothiocyanate (PITC) as the derivatizing agent. The present inventor published his first paper on the separation of amino acids using PITC in1993 (2)

For more than 50 years the clinical amino acid analysis of biological samples has been a problem and does not serve well clinical needs because of the long run times of 160 to 300 minutes per sample. Ion-exchange method is more widely used in clinical laboratories. Many of these laboratories still have the old Beckman ion exchange instrument (Beckman stopped the production of the instrument 20 years ago) and their run time per sample is 300 minutes. Recent commercial ion exchange method has a run time of 160 minutes per sample. Present commercial and in house HPLC methods have many other problems—the derivatization methods are complex, the derivatizing reagents and the derivatives are unstable, derivatization is not comprehensive, the instrumentation is sophisticated and very expensive, mobile phases are complex, hard to prepare, and also very expensive, and the complex gradient and column heating programs render trouble shooting peak resolutions very difficult. Two other major drawbacks of the present HPLC methods are 1) they can not provide a comprehensive amino acids profile using blood spot (critical for new born babies) and 2) do not estimate the total and or free concentrations of the two important thiol amino acids—Homocysteine and Cysteine as part of the profile. Although the two commercial methods—the ion-exchange method and PICO TAG method are “mature” and in use for more than 30 years the tests are very difficult to perform and highly skilled work force is a necessary. There is a lack of competition in the field for many decades and the cost of the assay is skyrocketing around the world. Six years ago the cost of one ion-exchange column was $3000 and in 2006 it was $5000. The present methods are uneconomical and drain our valuable resources—time and money. In 1993 “Waters” introduced a novel pre column derivatizing reagent (6-aminoquinolyl-N-hydroxysuccinimidyl carbamate (ACCQ TAG™) that reacts fast and in an elegant manner with amino acids (3) but it is a failure in the separation of all important amino acids in physiological samples. Little has happened in the last 14 years in spite of the innumerable varieties of analytical columns introduced by “Waters” to change the situation. The combined physical and chemical properties of the derivatives of amino acids in biological samples with ACCQ TAG are not amenable for the separation of important 27 amino acids. There has not been any break through in simplicity, sensitivity, robustness, and many other ideal features necessary for this clinical test to be successful for a long time. The test is a necessity but it is a big drag in a modern clinical laboratory.

It is well known that the best and simplest method for the analysis of amino acids is the pre-column derivatization method because of the short run times and ease of peak resolutions using reversed phase columns with high theoreretical plates. An ideal pre-column derivatizing agent should have the following characteristics: It is a stable compound, readily available, quickly (2-5 mins) reacts with amino acids (both primary and secondary) and forms only one derivative with each and every one of them, the derivatives are stable in solution, imparts good (femto mole) sensitivity for the assay, the derivatives have the optimal hydrophilic and hydrophobic characteristics to be amenable for the separation of all the important amino acids in a short time, and the derivatives are readily purified from excess reagent and side products of the reaction. Such a derivatizing agent (a fluorescent tag) was initially conceived more than 30 years ago by Dr. Perrett in England and it has been rediscovered by the present inventor and nicely exploited and proven to be the best for the analysis of amino acids profile in biological samples and it is the principal ingredient of this Patent application.

The reagent 1-Napthylisocyanate (NIC), was first used Amos Neidle and his co workers in 1989 (4).for the separation of 21 amino acids in 160 minutes (in addition there was a recycle time of 40 minutes before next injection). They barely recognized the importance and properties of the reagent NIC. The HPLC instrument used by them was quite antiquated and it lacked any modem features. The separation of the amino acid derivatives was not state of the art work even at the time. The work lacked precision and accuracy data on human plasma samples and normal values for healthy volunteers. Neither the original workers nor others who pursued work in the area recognized the great properties of the fluorescent tag and its amino acids derivatives until almost 20 years later by the present inventor.

### DESCRIPTION OF INVENTION

### Experimental Details for the Analysis of Free Amino Acids in Human Plasma Samples Using 1-Naphthylisocyanate

### Materials

Sodium dihydrogen phosphate, Sodium Hydroxide, Boric acid, Perchloric acid, Tris(carboxy ethylphosphine Hydrochloride), HPLC solvents acetonitrile, methanol, and water and Sodium heptane sulfonate were purchased from Fisher Scientific, Fair Lawn N.J. Amino acid standards (solids) were obtained from Sigma, St Louis, Mo.

### Preparation OF Reagents

1.0 M Borate buffer was prepared from boric acid and the pH was adjusted to 6.25 using sodium hydroxide solution.

Amino acid Standard: 400 μM/L standard was prepared by dissolving 0.1 mM amount of each amino acid (except—Homocystine and Cystine—only 0.05mM) were weighed and transferred to a 250 ml beaker, The amino acids were dissolved using minimum amount of 1M Sodium hydroxide solution and neutralized (using a pH meter and 1M hydrochloric acid solution). The neutral solution was transferred quantitatively to a 250 ml volumetric flask, the solution made up to volume and mixed well for uniform concentration. The solution was aliquoted and kept frozen in −80° C. freezer. The solution contained all amino acids including Glutamine, Glutamic acid, Aspartic acid, Aspargine and Tryptophan and was found to be stable for at least six months. The standard solution compared well with several of our own separate preparations and against external standard from Sigma Chemical Company.

Internal Standard: Both Nor Leucine and Nor Valine were found to be good for the work. 250 μM/L neutral solutions were separately prepared and aliquots were kept frozen at −80° C. Norvaline was used when Cystathionine had to be quantitated, Cystathionine elutes near Norleucine in our conditions of analysis.

Derivatization reagent: It was prepared by dissolving 10 μl of 1-Naphthylisocyanate in 4 ml of dry acetone in a test tube. The solution was prepared just prior to use.

### Equipment

The Hitachi HPLC system consisted of two (Model L2130) pumps, a built in Model L2200) degasser, an Auto sampler with Peltier cooling, a (Model L2300) Column oven, and a (Model L2480) Fluorescence detector with a 3 μl flow cell and a Dell Optiplex GX270T computer with EZchrom Elite software for data processing and control of all the instruments. The analytical column is a 3 micron C-18 ODS2, Hypersil column 15×0.46 cm (from Fisher-Thermo Electron, Bellefonte, Pa.)

A microcentrifuge (Eppendorf model 5418) was obtained from Fisher-Thermo Electron.

### Derivation

A. For plasma or Aqueous Standard

Pipetted 100 μl of water (blank) or aqueous standard or protein free filtrate (prepared using EDTA plasma and “Micron YM-10” filter device from Millipore, Mass.) into a 2 ml capacity labeled micro centrifuge tube with caps. To all tubes, 100 μl of internal standard, (Norleucine) 50 μl of borate buffer and 300 μl of HPLC grade water were added. The solutions in the centrifuge tube were gently mixed. 250 μl of derivatization reagent was added to each tube. Immediately after addition the tube was closed and the solution vortexed for few seconds. After a minute, the fine precipitate of 1,1′-dinaphthyl urea was filtered using a Whatman filter device (Mini UniPrep™ 1-5 ml volume, with nylon membrane, pore-2 μm) and the filtrate was extracted with 1 ml of Cyclohexane (to remove excess reagent and its hydrolytic product 1-Naphthylamine) and the top organic layer was removed under reduced pressure. Cyclohexane wash was repeated twice more and the aqueous layer stored at −4° C. until analysis. Analysis was performed diluting 40 μl of the solution with 200 μl of HPLC water and 3 μl injected into the column. Chromatograms of an aqueous standards, healthy volunteer plasma and reagent blank are shown in FIGS. 1A, 1B 2, & 3.

Alternative method for protein free filtrate: When 100 or 200 μl of plasma only was available, added corresponding amount of internal standard and equal volume (100 or 200 μl) of 6% perchloric acid to the plasma. The solutions were immediately mixed, left to stand for 5 minutes and then centrifuged. 200 or 300 μl of clear supernatant was transferred to a new micro centrifuge tube, neutralized and used for derivatization.

B. For Blood Spot

The derivatization procedure for the blood spots was similar to that for plasma. Two blood spots were obtained using ⅛^{th} inch diameter hand puncher. Amino acids were extracted by gentle tumbling using an aqueous organic mixture of 200 μl of methanol and 50 μl of an aqueous solution of internal standard (62.5 μM) solution) for 45 minutes using a 2 ml micro centrifuge tube. 150 μl of the top clear solution was transferred to a 13×75 mm glass test tube and the solution evaporated at 30° C. (oven) for 30 minutes. The residue was dissolved in 300 μl of HPLC water and treated with 25 μl of 1M borate buffer and 100 μl of the derivatizing reagent (which was obtained by diluting 1:4 the initial solution (refer above to derivatizing reagents preparation) with acetone. The standard for the blood spot work was processed as follows. Pipetted 3 μl of 400 μM/L aqueous standard solution used in the regular plasma work into a 2 ml micro centrifuge tube containing 200 μl of HPLC water and 50 μl of (62.5 μM/L) internal standard. The solutions were mixed and 150 μl of the solution was transferred to 13×75 mm glass test tube. To the standard solution, added 25 μl of 1M borate buffer (pH 6.25) 200 μl of HPLC water, and 100 μl of derivatizing reagent (refer above). After standing at room temperature for a minute with the derivatizing reagent the blood spot or the standard solution was centrifuged or filtered to remove the white precipitate and the clear top solution was transferred to new micro centrifuge tube (2 ml) and extracted 3 times with 0.5 ml cyclohexane (the top layer aspirated under reduced pressure). 3 μl of the derivatized solution was injected into the HPLC column. Chromatograms of an aqueous standard and a CDC blood spot control are shown in FIGS. 4 and 5.

Mobile phase (MP) A is a mixture of 20 mM phosphate buffer pH 5.9 and methanol 94:6 V/V containing 15 mg Sodium heptane sulfonate per 100 ml of solution.

Mobile phase (MP) B is a mixture of 20 mM phosphate buffer pH 5.73, methanol and acetonitrile 50:24:26 V/V containing 15 mg of Sodium heptane sulfonate per 100 ml of solution.

### Gradient Program and other Experimental Conditions for the Separation of Amino Acids

| | | | Flow/ | |
|---|---|---|---|---|
| Time | MP-A % | MP-B % | min | |
| 0 | 70 | 30 | 1.2 | Column oven at 41° C. and |
| | | | | auto sampler at 4° C. |
| 5.4 | 70 | 30 | 1.2 | The Fluorescence detector: |
| | | | | Excitation wavelength - 238 nm |
| 5.6 | 56 | 44 | 1.2 | and the emission wavelength - |
| | | | | 385 nm. |
| 7.8 | 56 | 44 | 1.2 | the photo multiplier of the |
| | | | | detector had 3 different |
| 8.0 | 46 | 54 | 1.3 | voltage settings -high, medium |
| | | | | and low. It was used at |
| 10.4 | 46 | 54 | 1.3 | the lowest voltage setting. |
| 10.6 | 20 | 80 | 1.3 | The volume of detector flow |
| | | | | cell was 3 μl. |
| 14.4 | 20 | 80 | 1.3 | Data collection was stopped at |
| | | | | 23 minutes |
| 14.6 | 0 | 100 | 1.3 | All segments of the gradient |
| | | | | were linear |
| 23 | 0 | 100 | 1.3 | |

The column was equilibrated at initial conditions for 10 minutes (cycle time) before next injection. Calculations were based on peak height determinations through out work

### Relative Fluorescence Response for Amino Acids Derivatives in the Gradient Method, Detection Limits, and Linearity

Based on the chromatogram of a derivatized aqueous standard of Amino acids for physiological samples the relative fluorescence response of all important amino acid derivatives were calculated using Valine peak height as the reference. The values are given in Table 1. The highest responses were noted for Aspartic acid, Glutamic acid and Alanine. The smallest responses were noted for Cystine and Tryptophan. The latter two derivatives have two Naphthyl carbamoyl groups per molecule and internal quenching decreases their fluorescence response considerably. Although such quenching occurs in Lysine and Ornithine, their fluorescence response is good and better than those for Cystine and Tryptophan and the former amino acids can be estimated by fluorescence detection. Except for the few above mentioned all other amino acids have approximately the same fluorescence responses

The smallest amount of sample and standard was used in the blood spot work. CDC has determined that the volume of plasma in two blood spots of ⅛^{th} inch diameter manual punch is 3.0 (±0.2%) μl. The final volume of derivatized solution in the blood spot (and the corresponding aqueous standard) work was about 300 μl and 3 μl was injected into the column.

The smallest concentration of aqueous standard used was 10 μM/L. The final volume of the derivatized solution was about 300 μl. 1 pico mole of each standard was injected into the column. Using the standard chromatogram and a signal to noise ratio of 3:1, the detection limits for the amino acids ranged from 40-950 femto moles. The smallest detection limit (40 femto moles) was calculated for Aspartic acid, Glutamine and Alanine and the highest detection limit (980 f moles) was noted for Tryptophan and the next higher detection limit was for Cystine.

### BRIEF SUMMARY OF THE INVENTION

More than 40 years since the beginning of amino acids profile analysis for clinical use, 1-Napthylisocyanate (NIC) has been found and proven as the best pre column derivatizing reagent. The fluorescent tag reacts quantitatively in one minute with all amino acids, and forms only one derivative with each one of the amino acids. The derivatives are very stable in solution for more than a day and longer (months/years) at lower temperatures (−4 to −80° C.). 35 amino acids (plus two internal standards and ammonia) in plasma have been separated in record shortest time of 20 minutes using a simple high pressure binary gradient, reversed phase (C-18, 3 micron, 150×4.6 mm) column, and a femto moles sensitive method. The challenge was accomplished with great success by optimization of several factors. The robustness of the method was established from linearity (0 to 1200 μM/L), precision (<6%), and accuracy (95%) studies and determination of plasma concentrations of free amino acids for few healthy volunteers and abnormal patients. For the first time a sample can be analyzed with in one hour of its arrival in the laboratory. 48 samples can be analyzed in 24 hours using one ($42,000) instrument. The method has two more unique, experimentally confirmed features unknown with any of the present amino acids profile methods. It provides: 1) a comprehensive amino acids (27) profile using a blood spot (3 μl of human plasma, the work was validated using “CDC” blood spot controls for two years) and 2) the free and total concentrations of Homocysteine and Cysteine as part of a full amino acids profile on human plasma. The following simple features of the method: 1 mobile phases, 2 traditional high pressure binary gradient system, 3 gradient program, 4 analytical column, 5 adaptation, 6 trouble shooting peak resolutions, 7 femto mole sensitivity, 8 very low cost of the instrument, column and reagents, 9 less than 20 minutes run time for a sample without temperature variations, 10 good precision (<6%) and accuracy (95%) of the method, 11 full plasma amino acid profile using one blood spot, 12 plasma amino acid profile that will include free and total concentrations of Homocysteine and Cysteine—qualify the method as the best choice for amino acids profile in biological samples and protein hydrolyzates in many decades. The method will lower health care costs by more than 60% in the area and a savings of at least $50 million (US) per year in the world. The method provides patient results in the shortest time in the history of the method and ensures quality patient care. The attributes of the method are due to the versatility of the derivatizing agent, properties of its amino acid derivatives and optimization of factors that influence their separation and detection.

TABLE 1
Comparative Fluorescence Response of Derivatized Amino Acids

| Amino Acid | Fluorescence Intensity |
|---|---|
| ASP | 2.0 |
| GLU | 2.1 |
| OH-PRO | 0.7 |
| ASN | 1.2 |
| SER | 1.0 |
| GLN | 1.1 |
| GLY | 1.2 |
| CIT | 1.0 |
| TAU | 0.9 |
| PRO | 0.45 |
| THR | 0.96 |
| ALA | 1.9 |
| HIS | 1.35 |
| AABU | 1.45 |
| CYS | 0.7 |
| TYR | 1.1 |
| ARG | 1.1 |
| HCY | 0.25 |
| VAL | 1.0 (Arbitrarily fixed) |
| MET | 0.7 |
| NVAL | 0.8 |
| ILEU | 1.47 |
| LEU | 1.32 |
| NLEU | 0.77 |
| PHE | 1.1 |
| TRP | 0.02 |
| (CYS)₂ | 0.35 |
| LYS | 0.72 |
| 0RN | 0.40 |

Values are for the same concentration of aqueous standard (except for NVAL and NLEU)

TABLE 2
Comparison of widely used HPLC Amino Acids Profile Methods for Biological Samples

| | NIC* | PITC** | OPA*** | ION-EXCHANGE**** |
|---|---|---|---|---|
| Derivatization method | Pre column | Pre column | Pre column | Post column |
| Derivatizing agent | Stable | Stable | Limited | Stable under Nitrogen |
| Derivatization time | <1 min. | 20 min | <1 min | Requires heating at 150° C. |
| Derivative stability | Stable in solution for one | Limited in solution | Limited, about 3 min. | Limited |
| | day, longer at −4-−80° C. | | | |
| Reaction products & | None | None | None | None |
| reagent interferences | | | | |
| Detection | Fluorescence | UV 254 nm | Fluorescence | At 2 wave lengths, 540 & 440 nm, |
| | | | | Sophisticated optics (expensive) |
| Sensitivity | Femto moles | Pico moles | Femto moles | Pico moles |
| # of compounds in profile | 39 (5 trace amino acids) | 26 | 38 (15 trace amino acids) | 41(12 trace & unimportant amino acids) |
| Reproducibility | Very good (<5%) | Good | Very good | Good |
| Adaptation & troubles | Simplest | Difficult | Very difficult | Most difficult |
| shooting peak resolutions | | | | |
| Instrument nature; cost | Simplest, $45000 | Simple ($60,000) | Sophisticated ($65,000) | Very sophisticated ($130,000) |
| Maintenance cost/year | $1000 (self) | $8000 (vendor) | $7000 (vendor) | $13000 (vendor) |
| Cost of analytical column | $450 | $800 | $550 | $5000 |
| Nature of mobile phases | Very simple | Simple | Complex | Most complex |
| Gradient | Binary gradient | Binary gradient | Tertiary | 5 buffers, |
| | In house preparation | From vendor | In house preparation | From vendor (1 liter costs about $100) |
| Run timer per sample | 20 + 10 min | 120 (vendor's method) | 65 min | 150-300 min |
| # of samples per day | 48 | 12 | 22 | 5-9 |
| Serious draw backs | None | Removes excess reagent | Cystine, Proline & hy- | Does not serve clinical needs well |
| | | using special vacuum | droxy Proline not | Most expensive in all respects |
| | | set up. | estimated | Hampered research, and governmental |
| | | | Peaks resolutions big | regulations of feed industry etc |
| | | | problem | |
| Comprehensive profile | §Easily & nicely done | Impossible, poor method | Not done, Not compre- | Impossible, poor method sensitivity |
| using one blood spot¶ | | sensitivity | hensive | |
| Estimation of plasma | §Can be done by a | Impossible poor method | Not done | Impossible, poor method sensitivity |
| free Hcy & Cys as | simple method | sensitivity | | |
| part of full profile. | | | | |
| Savings in health care | Estimated to be about | 10-20% increase of | 10-20% of $5 million | 10-20% increase of $75 million. |
| and related areas/year | $60 million. | $15 million | | |

*1-Naphthylisocyanate
**PITC—Phenylisothiocyanate,
***OPA—orthoPhthalaldehyde,
****uses Ninhydrin -coloring reagent
¶3 μl plasma.
§First time introductions in the history of the method

TABLE 3
Comparison between Neidle's and the Present Method

| Subject | Neidle's 1989 Method | Inventor's Method (2007) |
|---|---|---|
| Column | 5 micron, 250 × 4.6 mm, C-18, Long column | 3 micron, 150 × 4.6 mm, C-18, Short |
| | Low theoretical plates, Long run times | column, higher theoretical plates |
| | | Shorter run times |
| Gradient | Binary, low pressure gradient | Binary high pressure gradient |
| | single pump | 2 pumps |
| MPs^{§} | 20 mM, (1:1) acetate-phosphate mixture | 20 mM, phosphate, Buffer A pH 5.9 |
| | Both buffers pH 5.4 (bad choice) | Buffer B pH 5.73; MP-A 94:6 |
| | MP-A- 85:12.5:2.5 (buffer:methanol: | (buffer:methanol), MP-B (50:24:26) |
| | ^{$}ACN); MP-B- 55:45 buffer:ACN | buffer:ACN:methanol |
| Instrument | Poor set up, flow cell volume unknown | State of the art, 3 μl flow cell |
| Detector | Fixed voltage multiplier | Variable voltage multiplier |
| Column | Room temp | 42 ± 2° C. |
| Temp | | |
| Ion pairing | No (great flaw) | Yes, to both MPs, 150 mg/L |
| reagent | | |
| Number of | ^{@}20, omitted-OHPRO, CIT, ASN, TAU | 38, includes HCY, CYS, ALILEU & |
| Amino acids | ASN, GLN, ORN* | CYSTA* |
| in standard | | |
| Run time | 160 + 40 min. | ¶20 + 10 min. Shortest in history |
| Blood spot | Not done | ¶Comprehensive profile −3 μl plasma |
| Analysis | | “CDC” quality controls for 2 years |
| HCY, CYS* | Not done | ¶Easily done by a simple method. |
| estimation | | |
| Clinical use | No one uses. (200 min. between samples) | Best in history (4 decades) |
| Sensitivity | 4 pico mole (20-30 μl injection) | 0.5 pico mole (3 μl injection) |
| Precision | Aqueous standard (n = 6) | plasma, Mean <6% (n = 16) |
| Accuracy | Not done | Mean 95% (plasma, n = 8) |
| Linearity | Not known | 0 to 1200 μM/L |

^{§}MP—mobile phase,
*refer Table 4 for abbreviations,
^{$}ACN Acetonitrile,
^{@}Authors didn't have a reliable standard.
¶Historical features

TABLE 4
Retention Times of Peaks and Abbreviations
used in Text and Figures

| Abbreviations | # | Amino Acid | Retention Times (mins) |
|---|---|---|---|
| P-SER | 1 | Phosphoserine | 1.96 |
| ASP | 2 | Aspartic Acid | 2.23 |
| GLU | 3 | Glutamic Acid | 2.51 |
| HOPRO | 4 | Hydroxy Proline | 2.74 |
| AAAA | 5 | £Amino Adipic Acid | 3.12 |
| SAR | 6 | Sarcosine | 4.43 |
| ASN | 7 | Aspargine | 4.63 |
| PEA | 8 | Phospho Ethanol Amine | 4.82 |
| SER | 9 | Serine | 5.22 |
| GLN | 10 | Glutamine | 5.43 |
| GLY | 11 | Glycine | 5.64 |
| CIT | 12 | Citrulline | 6.06 |
| TAU | 13 | Taurine | 6.30 |
| PRO | 14 | Proline | 6.58 |
| THR | 15 | Threonine | 6.90 |
| ALA | 16 | Alanine | 7.17 |
| HIS | 17 | Histidine | 7.74 |
| 3-Me HIS | 18 | 3-MethylHistidine | 8.36 |
| CYS | 19 | Cysteine | 8.58 |
| AABU | 20 | £-AminoButyricAcid | 8.78 |
| TYR | 21 | Tyrosine | 9.06 |
| ARG | 22 | Arginine | 9.38 |
| HCY | 23 | Homomocysteine | 10.92 |
| AMMO | 24 | Ammonia | 11.19 |
| ETA | 25 | Ethanol Amine | 11.55 |
| VAL | 26 | Valine | 11.85 |
| MET | 27 | Methionine | 12.28 |
| NORVAL | 28 | NorValine | 12.50 ¶ |
| ILEU | 29 | Isoleucine | 14.52 |
| ALISOLEU | 30 | Alloisoleucine | 14.68 |
| LEU | 31 | Leucine | 14.93 |
| NORLEU | 32 | Norleucine | 15.37 ¶ |
| CYSTA | 33 | Cystathionine | 15.50 |
| PHE | 34 | Phenylalanine | 15.60 |
| TRP | 35 | Tryptophan | 15.90 |
| (CYS)₂ | 36 | Cystine | 16.50 |
| HOLYS-1 | 37 | Hydroxylysine-1 | 17.33 |
| HOLYS-2 | 38 | Hydroxylysine-2 | 17.56 |
| ORN | 39 | Ornithine | 18.02 |
| LYS | 40 | Lysine | 18.49 |

The retention times of peaks vary with different column lots, mobile phase composition, gradient etc., but the order of elution is the same.
¶Internal Standard. All the 40 amino acids can be separated by the gradient method used in this work.

TABLE 5
Detection Limits of 1-Naphthyl Carbamoyl Amino Acids

| | Detection Limit (femto moles) |
|---|---|
| Amino Acid | Calculated using 1 pico mole injection |
| ASP | 40 |
| GLU | 40 |
| OH-PRO | 122 |
| ASN | 82 |
| SER | 77 |
| GLN | 90 |
| GLY | 84 |
| CIT | 97 |
| TAU | 108 |
| PRO | 226 |
| THR | 103 |
| ALA | 46 |
| HIS | 80 |
| AABU | 38 |
| TYR | 93 |
| ARG | 82 |
| VAL | 80 |
| MET | 124 |
| ILEU | 65 |
| LEU | 71 |
| PHE | 93 |
| TRP | 980 |
| (CYS)₂ | 850 |
| LYS | 120 |
| ORN | 230 |

TABLE 6
Intra Assay Precision Data for the Assay
using Patients' Plasma Pool

| | Compound | Mean* ± S.D. | C.V. |
|---|---|---|---|
| 1. | ASP | 26.5 ± 0.7 | 2.6 |
| 2. | GLU | 83.3 ± 2.0 | 2.4 |
| 3. | OH-PRO | 7.3 ± 0.7 | 1.0 |
| 4. | ASN | 67 ± 1.6 | 2.4 |
| 5. | SER | 114 ± 2.3 | 2.0 |
| 6. | GLN | 862 ± 14 | 1.6 |
| 7. | GLY | 247 ± 4.2 | 1.7 |
| 8. | CIT | 49 ± 1.3 | 2.7 |
| 9. | TAU | 99 ± 2.8 | 2.8 |
| 10 | PRO | 290 ± 10 | 3.4 |
| 11 | THR | 128 ± 3.8 | 3.0 |
| 12 | ALA | 494 ± 42 | 8.4 |
| 13 | HIS | 96 ± 4.4 | 4.6 |
| 14 | AABU | 20 ± 3.0 | 15 |
| 15 | TYR | 97 ± 3.9 | 4.0 |
| 16 | ARG | 148 ± 4.9 | 3.3 |
| 17 | VAL | 337 ± 9.9 | 2.9 |
| 18 | MET | 37 ± 1.2 | 3.1 |
| 19 | ILEU | 117 ± 3.7 | 3.1 |
| 20 | LEU | 196 ± 4.3 | 2.2 |
| 21 | PHE | 85 ± 1.9 | 2.2 |
| 22 | 0RN | 59 ± 5.6 | 9.5 |
| 23 | LYS | 104 ± 5.8 | 5.5 |

*N = 6

TABLE 7
Inter Assay Precision Data for the
Assay Using Patient Plasma Pool

| Compound | Mean* ± S.D. | C.V |
|---|---|---|
| ASP | 19.6 ± 2.7 | 13.8 |
| GLU | 186 ± 12 | 6.5 |
| OHPRO | 7 ± 0.7 | 10 |
| ASN | 36 ± 2.3 | 6.4 |
| SER | 121 ± 8.2 | 6.8 |
| GLN | 304 ± 20.5 | 6.7 |
| GLY | 250 ± 14 | 5.5 |
| CIT | 31 ± 1.8 | 5.8 |
| TAU | 38 ± 2.2 | 5.8 |
| PRO | 310 ± 21 | 6.8 |
| THRE | 115 ± 10 | 9.0 |
| ALA | 316 ± 24 | 7.5 |
| HIS | 66 ± 2.9 | 4.4 |
| AABU | 15 ± 1 | 8.3 |
| TYR | 76 ± 4.7 | 6.2 |
| ARG | 72 ± 2.6 | 3.6 |
| VAL | 159 ± 5.5 | 3.5 |
| MET | 22 ± 1 | 4.6 |
| ILE | 53 ± 2 | 3.6 |
| LEU | 80 ± 3 | 4.1 |
| PHE | 61 ± 2 | 3.8 |
| LYS | 33 ± 4 | 11.5 |
| ORN | 95 ± 12 | 12.5 |

*n = 16

TABLE 8
Recovery Data for the Plasma Amino Acids HPLC Assay

| AMINO ACID | MEAN (μM/L)* | RECOVERY % |
|---|---|---|
| ASP | 8 | 90 |
| GLU | 93 | 112 |
| HOPRO | 5 | 109 |
| ASN | 44 | 101 |
| SER | 68 | 99 |
| GLN | 582 | 92 |
| GLY | 170 | 99 |
| CIT | 48 | 100 |
| TAU | 30 | 100 |
| PRO | 241 | 98 |
| THR | 91 | 98 |
| ALA | 327 | 97 |
| HIS | 68 | 97 |
| AABU | 12 | 97 |
| TYR | 61 | 102 |
| ARG | 75 | 96 |
| VAL | 263 | 105 |
| MET | 18 | 111 |
| ILE | 71 | 99 |
| LEU | 133 | 101 |
| PHE | 52 | 103 |
| TRP | 15 | 109 |
| (CYS)₂ | 0 | 102 |
| ORN | 55 | 103 |
| LYS | 185 | 115 |

• N = 8 (concentrations added ranged from 25 to 400 μM/L)

TABLE 9
Normal Patients'* Plasma Free Amino
Acids Concentrations by the New Method

| Amino Acid | Range (μM/L) | Mean¶ ± S.D (μM/L) |
|---|---|---|
| ASP | 3-16 | 9 ± 3 |
| GLU | 10-223 | 65 ± 56 |
| OHPRO | 5-23 | 11 ± 5 |
| ASN | 41-73 | 55 ± 11 |
| SER | 65-166 | 107 ± 32 |
| GLN | 550-1414 | 846 ± 272 |
| GLY | 170-559 | 307 ± 124 |
| CIT | 25-55 | 40 ± 8 |
| TAU | 22-107 | 42 ± 25 |
| PRO | 155-361 | 262 ± 68 |
| THR | 77-225 | 145 ± 52 |
| ALA | 256-644 | 450 ± 118 |
| HIS | 36-116 | 77 ± 21 |
| AABU | 9-28 | 17 ± 6 |
| TYR | 52-105 | 67 ± 15 |
| ARG | 53-167 | 89 ± 29 |
| VAL | 117-283 | 218 ± 45 |
| MET | 12-38 | 25 ± 7 |
| ILE | 29-126 | 62 ± 23 |
| LEU | 84-222 | 115 ± 41 |
| PHE | 47-96 | 59 ± 14 |
| TRP | 27-140 | 62 ± 36 |
| ORN | 41-77 | 65 ± 18 |
| LYS | 125-199 | 176 ± 29 |

*Adults ages 20-70
¶n = 12

TABLE 10
CDC Blood Spot Quality Control Samples Precision Data

| Compound | Mean ± S.D*. (μM/L) | C.V. % |
|---|---|---|
| ASP | 71 ± 4.7 | 6.6 |
| GLU | 635 ± 24 | 3.8 |
| OHPRO | 35 ± 1.9 | 5.4 |
| ASN | 144 ± 0.6 | 0.4 |
| SER | 459 ± 9.4 | 1.4 |
| GLN | 242 ± 1.8 | 0.8 |
| GLY | 1485 ± 8 | 0.5 |
| CIT | 72 ± 1.1 | 1.5 |
| TAU | 105 ± 1.2 | 1.5 |
| PRO | 540 ± 3.8 | 0.7 |
| THR | 351 ± 3.0 | 0.7 |
| ALA | 1482 ± 30 | 2.0 |
| HIS | 208 ± 1.6 | 0.8 |
| AABU | 30 ± 0.9 | 3.0 |
| TYR | 154 ± 1.4 | 0.9 |
| ARG | 136 ± 1.4 | 0.9 |
| VAL | 416 ± 9.0 | 2.0 |
| MET | 37.6 ± 0.8 | 2.0 |
| ILEU | 141 ± 0.1 | 0.7 |
| LEU | 410 ± 1.9 | 0.5 |
| PHE | 206 ± 0.8 | 1.9 |
| ORN | 588 ± 3.6 | 0.6 |
| LYS | 463 ± 6.0 | 1.3 |

N = 5 Intra assay

Data for the Chromatogram of Normal Patient Plasma—FIG. 2

| Name | Time | Height | Amount |
|---|---|---|---|
| | 1.480 | 30028 | 0.000 |
| ASP | 2.357 | 410606 | 39.306 |
| GLU | 2.593 | 1267073 | 139.849 |
| HOPRO | 3.083 | 65611 | 18.654 |
| ASN | 4.693 | 495869 | 85.789 |
| SER | 5.320 | 1171256 | 176.855 |
| GLN | 5.590 | 4263515 | 787.823 |
| GLY | 5.843 | 1986931 | 341.731 |
| CIT | 6.467 | 192807 | 34.354 |
| TAU | 6.657 | 1231934 | 260.441 |
| PRO | 7.080 | 715582 | 323.378 |
| THR | 7.460 | 747670 | 168.491 |
| ALA | 7.983 | 3069817 | 403.595 |
| | 8.343 | 25651 | 0.000 |
| HIS | 8.590 | 564706 | 82.030 |
| | 8.943 | 16258 | 0.000 |
| 3-Me-His | 9.280 | 23386 | 7.516 |
| AABU | 9.520 | 147891 | 14.009 |
| TYR | 9.713 | 646399 | 77.544 |
| ARG | 10.007 | 579530 | 68.460 |
| AMMO | 11.987 | 1373272 | 0.000 |
| VAL | 12.360 | 2090094 | 269.451 |
| MET | 12.610 | 315000 | 36.503 |
| | 13.020 | 103819 | 0.000 |
| | 13.557 | 414034 | 0.000 |
| ILE | 14.510 | 670135 | 64.497 |
| LEU | 14.857 | 1198780 | 131.939 |
| NLEU | 15.260 | 2210706 | 1.000 |
| PHE | 15.553 | 516235 | 62.652 |
| TRP | 15.997 | 51347 | 48.104 |
| | 16.287 | 16558 | 0.000 |
| HCY)2 | | | 0.000 BDL |
| (CYS)2 | | | 0.000 BDL |
| ORN | 18.713 | 468982 | 85.658 |
| LYS | 19.270 | 627025 | 222.599 |

Data for the Chromatogram of CDC Blood Spot Standard—FIG. 4

| Name | Time | Height | Amount |
|---|---|---|---|
| | 2.173 | 25520 | 0.000 |
| ASP | 2.337 | 1179106 | 385.798 |
| GLU | 2.577 | 1518642 | 366.603 |
| HOPRO | 3.087 | 381078 | 398.101 |
| ASN | 4.693 | 617038 | 392.371 |
| SER | 5.330 | 686246 | 389.743 |
| GLN | 5.587 | 321154 | 129.713 |
| GLY | 5.847 | 609004 | 397.780 |
| CIT | 6.357 | 589942 | 395.655 |
| TAU | 6.660 | 521186 | 400.698 |
| PRO | 6.990 | 272976 | 393.923 |
| THR | 7.343 | 591253 | 386.517 |
| ALA | 7.873 | 832071 | 376.946 |
| HIS | 8.453 | 766091 | 399.340 |
| 3-Me-His | | | 0.000 BDL |
| AABU | 9.620 | 838225 | 401.792 |
| TYR | 9.960 | 732363 | 393.186 |
| ARG | 10.230 | 920014 | 401.530 |
| AMMO | 12.327 | 379786 | 0.000 |
| VAL | 12.623 | 1133543 | 392.682 |
| MET | 12.873 | 948006 | 402.174 |
| ILE | 14.760 | 1106968 | 401.330 |
| LEU | 15.100 | 950670 | 399.831 |
| NLEU | 15.490 | 1011302 | 1.000 |
| PHE | 15.777 | 869733 | 401.592 |
| TRP | 16.113 | 133190 | 400.476 |
| HCY)2 | | | 0.000 BDL |
| (CYS)2 | 17.047 | 217722 | 188.434 |
| ORN | 18.637 | 548356 | 363.120 |
| LYS | 19.160 | 383326 | 352.950 |

Data for the Chromatogram of CDC Blood Spot Quality Control—FIG. 5.

| Name | Time | Height | Amount |
|---|---|---|---|
| | 1.400 | 70877 | 0.000 |
| | 1.523 | 43567 | 0.000 |
| | 2.147 | 156236 | 0.000 |
| ASP | 2.327 | 469851 | 72.120 |
| GLU | 2.580 | 5730095 | 648.916 |
| HOPRO | 3.083 | 73931 | 36.232 |
| ASN | 4.680 | 480961 | 143.477 |
| SER | 5.307 | 1686223 | 449.262 |
| GLN | 5.563 | 1260951 | 238.920 |
| GLY | 5.820 | 4808987 | 1473.542 |
| CIT | 6.327 | 222800 | 70.098 |
| TAU | 6.627 | 285342 | 102.914 |
| PRO | 6.950 | 788780 | 533.985 |
| THR | 7.300 | 1142510 | 350.382 |
| ALA | 7.803 | 6911331 | 1468.815 |
| HIS | 8.447 | 849032 | 207.622 |
| 3-Me-His | 8.763 | 37053 | 12.212 |
| | 9.173 | 24379 | 0.000 |
| AABU | 9.607 | 138867 | 31.227 |
| TYR | 9.960 | 610537 | 153.769 |
| ARG | 10.233 | 71672 | 14.674 |
| AMMO | 12.317 | 264931 | 0.000 |
| VAL | 12.613 | 2579630 | 419.225 |
| MET | 12.857 | 186002 | 37.018 |
| | 14.337 | 17651 | 0.000 |
| ILE | 14.763 | 840296 | 142.917 |
| LEU | 15.103 | 2075385 | 409.479 |
| NLEU | 15.497 | 2155731 | 1.000 |
| PHE | 15.783 | 949694 | 205.717 |
| TRP | | | 0.000 BDL |
| HCY)2 | | | 0.000 BDL |
| (CYS)2 | | | 0.000 BDL |
| | 17.883 | 12214 | 0.000 |
| ORN | 18.660 | 1880604 | 584.214 |
| LYS | 19.187 | 1060261 | 457.978 |

### DESCRIPTION OF FIGURES

Total number of Figures, is 10. For abbreviations please refer Table 4, the Y axis in all figures corresponds to micro volts—mv and X axis corresponds to time in minutes

FIG. 1A

The chromatogram shows the separation of all important amino acids (27+ammonia) in an aqueous neutral standard solution. It highlights the following good features: 1 only one derivative is formed for each amino acid, 2 the Naphthyl carbamoyl derivatives have the best properties for separation, 3 proves the good experimental conditions for their separation and 4 an idea of the fluorescence response of different amino acids derivatives. The figure is historical because the separation is good, comprehensive and fastest in the history. The concentration of all amino acids is 400 μM/L, except PSER & AABU (25 μM/L each), NLEU—the internal standard (250 μM/L), (CYS)₂—200 μM/L and AMMO—is an impurity.

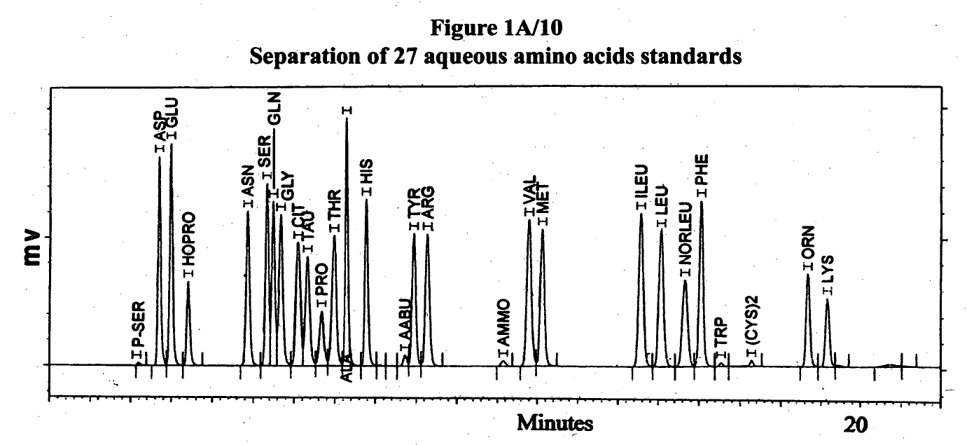

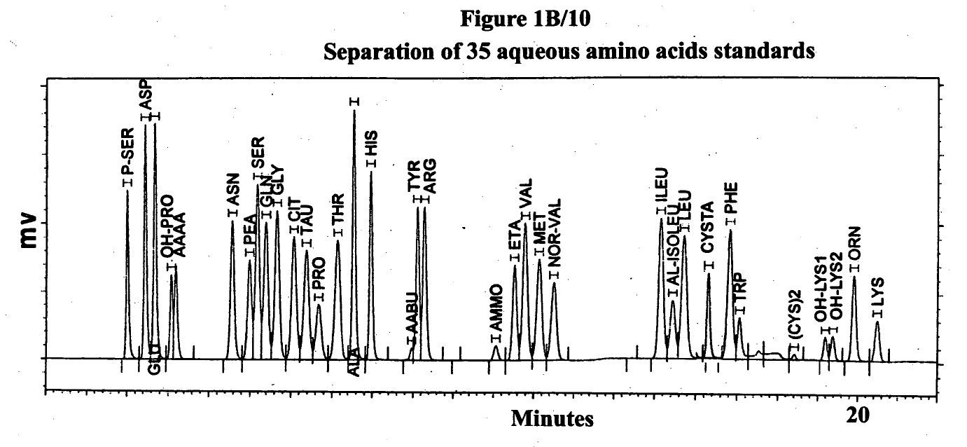

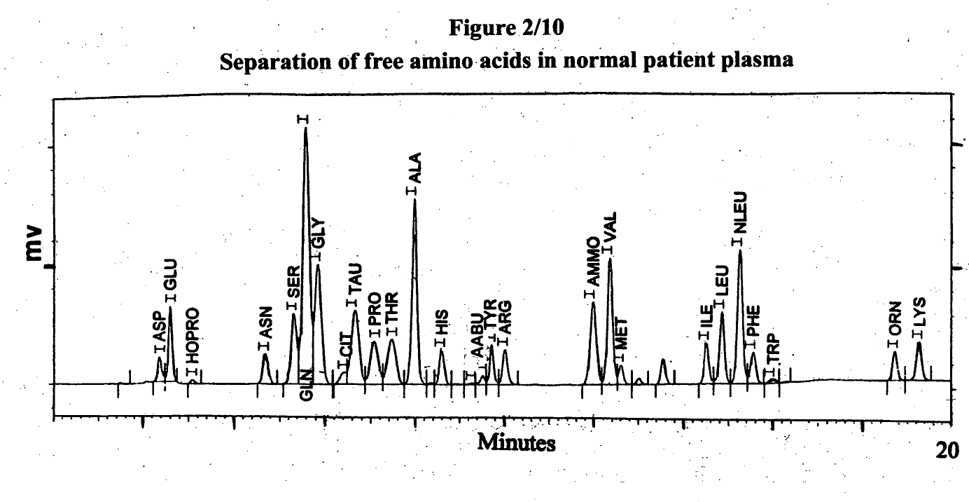

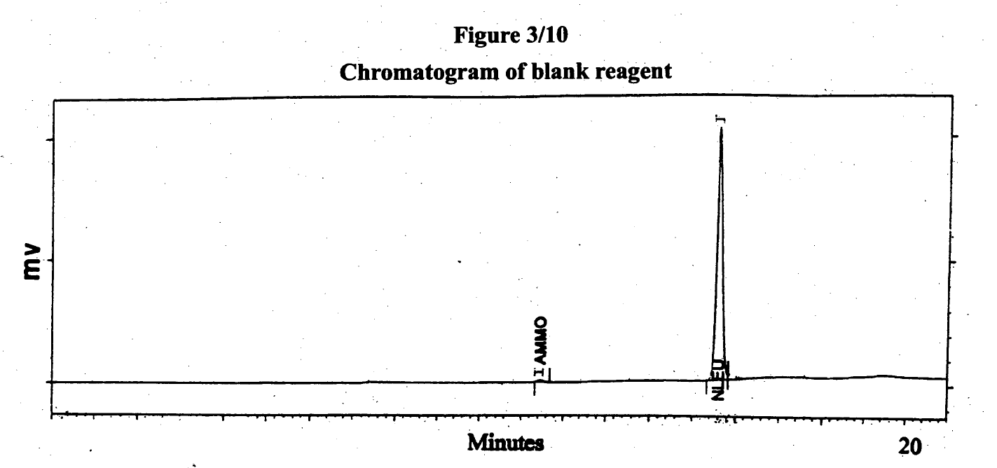

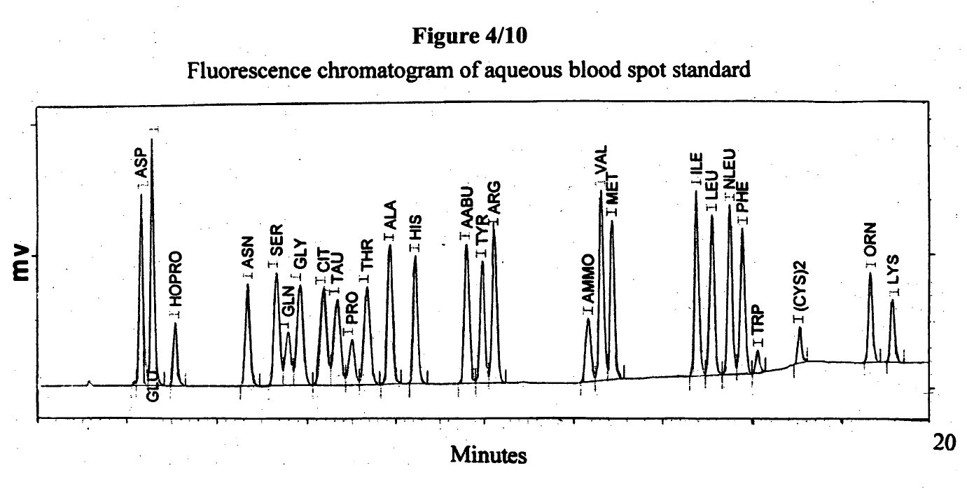

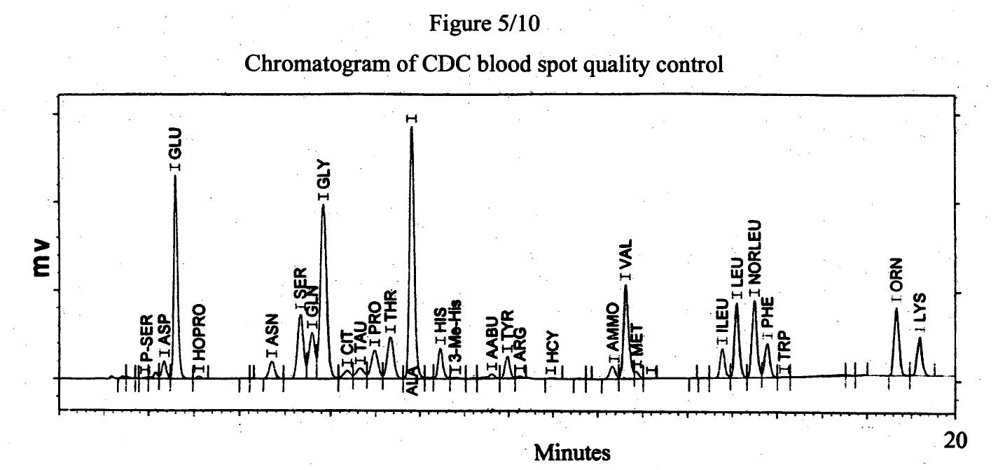

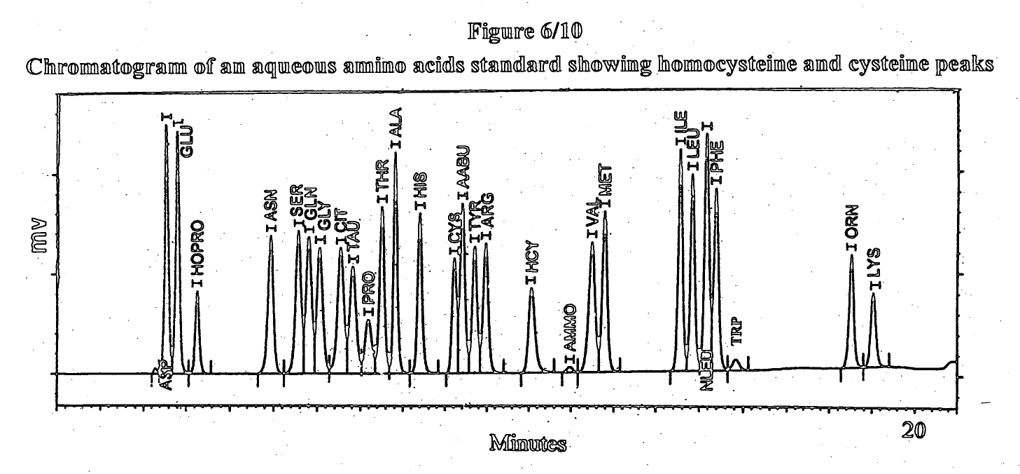

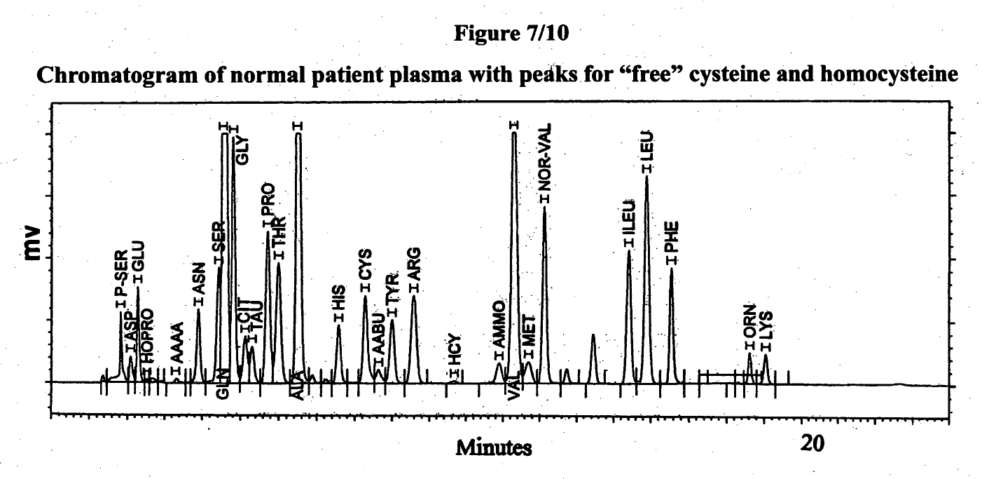

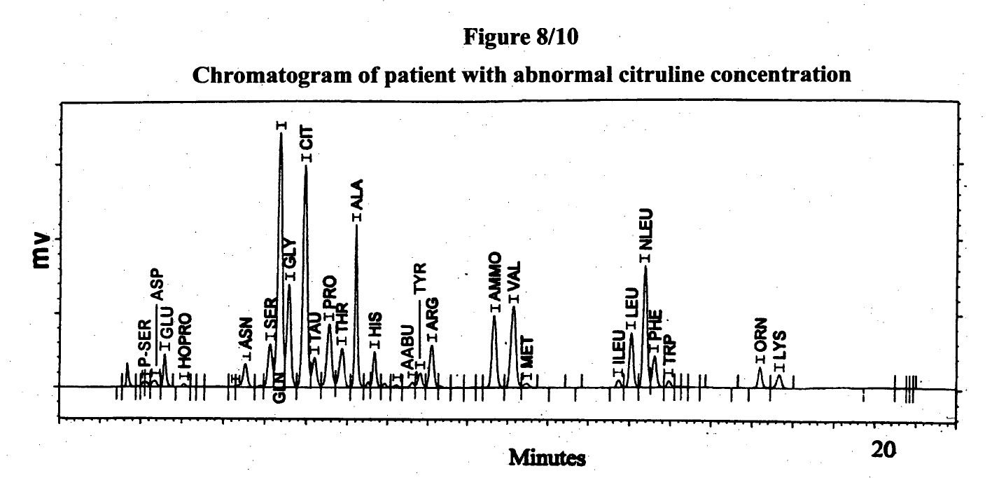

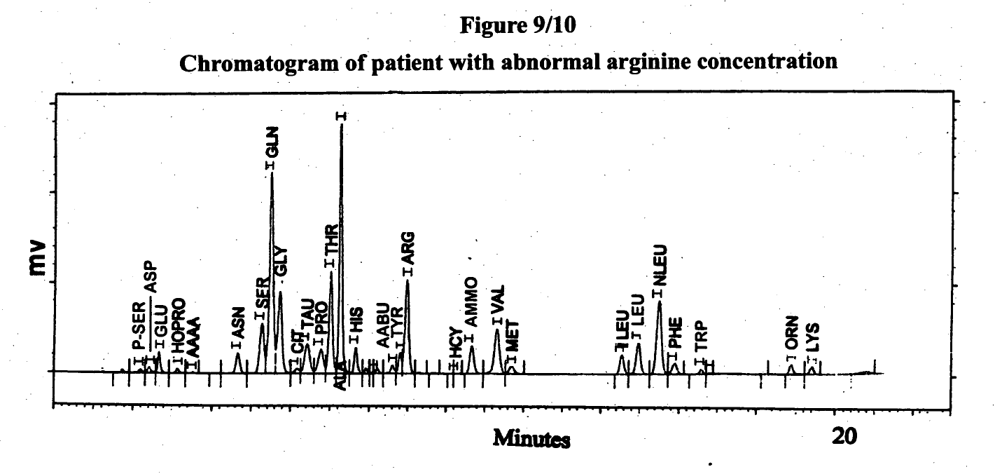

FIG. 1B

The figure shows the separation of an aqueous neutral solution of 35 amino acids (and AMMO) in record short time of about 20 minutes. FIGS. 1A & B demonstrates that old traditional way of preparing aqueous standards in 0.1N HCl solution is not best method since GLN,GLU, ASN,ASP,TRP and MET concentrations change with time even at −80° C. (refer FIG. 4). Our standards in neutral pH are stable for at least 6 months. The separation and retention times of HCY & CYS can be seen in FIGS. 6 & 7. The internal standard is NVAL. The total number of compounds that can be separated by the method is 35 plus NLEU, CYS, HCY. AAAA is a trace amino acid (concentration <10 μM/L, in normal patients). Because of their high concentrations (400 μM/L) the two peaks for OH-PRO and AAAA are not well separated. This figure also reflects good experimental conditions for the derivatization and separation of amino acids. The gradient is modified in the last two segments for this chromatogram as follows. From 10.6 to 14.4 mins. the gradient is 32% A and from 14.6 to 23 min. the gradient is 0% A. This change improves the resolution of 5 peaks from ILEU to TRP. The gradient change is used only when ALISOLEU and CYSTA are to be estimated.

FIG. 2

Shows the Fluorescence chromatogram of a healthy adult. The two peaks at 13.02 and 13.56 minutes are unidentified peaks. Analysis report for the sample is on page 42.

FIG. 3

Shows the fluorescent chromatogram of blank reagent using water (instead of sample or standard) and internal standard. It is a nice reflection of the absence of any interference from reagents in the assay.

FIG. 4

Shows the chromatogram of our aqueous standard (400 μM/L) for blood spot amino acids profile using 3 μl of the solution. This old standard used was prepared in 0.1 normal hydrochloric acid. The concentrations of GLN and GLU are not 400 μM/L. GLN is hydrolyzed in acid medium (0.1M HCl) to GLU. The standard was used for the estimation of amino acids other than GLN, GLU, ASN, ASP and TRP in CDC quality control blood spots. Analysis report for the standard against another 400 μM/L standard is given on page 46

FIG. 5

Shows the Chromatogram of a CDC blood spot quality control sample. The figure shows the nice separation of 27 amino acids, ammonia, and the internal standard NLEU. This figure shows the estimation of all important amino acids using 3 μl of plasma sample. The total volume of the derivatized solution will be about 250-300 μl, of which 3 μl is injected into the column with the detector photo multiplier set at the lowest voltage setting. The analysis report for the sample is given on page 47.

FIG. 6

This is the first fluorescence chromatogram of aqueous amino acids profile standard (400 μm/L) with peaks for “free” Cysteine (CYS) and Homocysteine (HCY) and has a run time of about 20 minutes. (Refer claim 5). The retention time for CYS is 9.2 min and that for HCY is 11.1 min. In this Fig CYS and AABU peaks are some what close due to the high concentration of AABU (400 μM/L). Normal concentration range for AABU for healthy person is 15-28 μM/L. This figure illustrates two important facts: 1) 1-Napthylisocyanate is the best pre column derivatizing agent for human plasma amino acids profile, because it facilitates the simple reduction of the preformed derivatives of Cystine and Homocystine and 2) our carefully optimized separation and detection experimental conditions are one of the best and they facilitate the appearance CYS and HCY peaks in very nice positions in the chromatogram. The profile chromatogram is the best in the clinical amino acids analyses history.

FIG. 7

It is the first free amino acids profile chromatogram of a normal patient's plasma sample with peaks for free Cysteine (CYS) and Homocysteine (HCY). (Refer claim 5) with a run time of about 20 minutes. The two peaks appear in positions where there is no interference from other amino acids in plasma. The figure illustrates the versatility and superiority of the derivatizing agent NIC and its amino acids derivatives and our carefully optimized chromatographic conditions for the profile analysis.

FIG. 8

It is the fluorescence chromatogram of patient plasma with abnormal Citrulline concentration. Citrullineamia patient. The main peak of interest in this Figure is the CIT peak.

FIG. 9

It is the fluorescence chromatogram of a patient with abnormal Arginine concentration. Arginase enzyme deficiency patient. The main peak of interest in this Fig is the ARG peak

### IDENTIFICATION OF DRAWINGS

### Please Refer Table 4 for Abbreviations

All FIGS. from 2 to 9 are replacement figures. FIG. 1A and 1B are new and annotated

New FIG. 1A is the same as the old FIG. 1A, except that it is for the modified new standard. The concentration of AABU in the new standard is 25 μM/L and this covers the range 9-28 μM/L for normal patients. This change helps good separation of CYS peak eluting before AABU (Refer Table 4 and FIGS. 6 & 7).

New FIG. 1B has three additional peaks compared to the old FIG. 1B. The three peaks are for: 1 £-Amino adipic acid (AAAA), 2 Ethanol amine (ETA), and 3 Cystathionine. (CYSTA). All 3 compounds are present in trace amounts (0 to 10 μM/L) in healthy adults. CYSTA is elevated in vitamin deficiencies. The new Fig illustrates that the method can be easily expanded to include additional trace amino acids. But the main focus is the separation of 27 amino acids in FIG 1A. The separation of 38-40 compounds (Table 4) with in 20 minutes by a simple and robust method is a clear reflection of the versatility of the derivatizing agent and the separation method developed in this work.

### LITERATURE REFERENCES

1. Steve A. Cohen et al. The PICO Tag™ Method—A manual of advanced techniques for amino acids analysis Waters Chromatography, Bedford, Mass. 1989
2. Hariharan M, Sundar Naga and Ted Van Noord. Systematic approach to the development of plasma amino acids analysis by high performance liquid chromatography with ultra violet detection and pre column derivatization using Phenylisothiocyanate J. Chromatogr. B 621 (1993) 15-22
3. Steven A. Cohen and Dennis P. Machaud. Synthesis of a Fluorescent Reagent, 6-Amino quinoyl-N-Hydroxysuccinimidyl Carbamate, and its Application for the analysis of Hydrolysate Amino Acids via High Performance Liquid Chromatography. Anal. Biochem 211(1993) 279-87
4. Amos Neidle, Miriam Banay-Schwatrz, Shirley Sacks and David S. Dunlop. Anal. Biochem 180 (1989) 291-97
5. Durk Fekkes, Astrid van Dalen, Margriet Edleman, Ans Voskuilen. Validation of the determination of amino acids in plasma by high performance liquid chromatography using automated pre column derivatization with ortho-phthaladehyde. J. Chromatogr B 869 (1995) 177-186
6. Durk Fekkes. Review State of the art of high performance liquid chromatographic analysis of amino acids in physiological samples J. Chromatogr B 682 (1996) 3-22

I claim

1. I claim the successful development of the first, most simple, robust, economical, and femto mole sensitive, reversed phase, high pressure binary gradient liquid chromatographic method, using 1-Naphthylisocyante as the pre column fluorescent derivatizing reagent, to separate and estimate the concentrations of 38 amino acids in human biological samples in about 20 minutes by prudent optimization of many separation and detection factors.
Success claim 1 depends on the factors
2 1-Napthylisocyanate said pre column derivatizing reagent, its properties and properties of its amino acids derivatives.
3 The C-18, silica, packing material for the reversed phase analytical column, the 3 micron, particle size of the column material, ODS-2, Hypersil, brand of the column material, and dimensions of the column 150×46 mm.
4 The nature of the buffer, Monobasic Sodium dihydrogen phosphate, its concentration, pH and the percentage composition of organic solvents in the two mobile phases A and B used in gradient separation.
5 The nature and concentration of ion pairing agent, Sodium heptane sulfonate added to both mobile phases A and B.
6 The temperature of the analytical column.
7 The gradient program for the separation.
8 The traditional liquid chromatography instrument, consisting of binary high pressure gradient pumps, built in degasser, column oven, auto sampler, fluorescence detector with a 3 μl flow cell and optimized critical sample flow path.
Explanations of the factors
